(19) United States
(12) Patent Application Publication
(10) Pub. No.: US 2023/0397612 A1
(43) Pub. Date: Dec. 14, 2023

(54) miRNA, and Derivative Thereof and Use Thereof

(71) Applicant: South China Normal University, Guangzhou (CN)

(72) Inventors: Sichun ZHENG, Guangzhou (CN); Yiguang LIN, Guangzhou (CN); Huijun JI, Guangzhou (CN)

(21) Appl. No.: 18/096,538

(22) Filed: Jan. 12, 2023

(30) Foreign Application Priority Data
Jan. 20, 2022 (CN) 202210068143.0

(51) Int. Cl. A01N 63/60; C12N 15/113; A01P 7/04; A01N 25/30; A01N 25/06

(52) U.S. Cl. CPC A01N 63/60; C12N 15/113; A01P 7/04; A01N 25/30; A01N 25/06; C12N 2310/141; C12N 2330/51

(57) ABSTRACT

A miRNA, derivative and use thereof are disclosed. The miRNA comprises a nucleic acid sequence as shown in SEQ ID NO. 1 or a nucleic acid sequence obtained by modifying, substituting, deleting or adding at least one base to the nucleic acid sequence as shown in SEQ ID NO. 1. According to the disclosure, the used miRNA is derived from Glycine max, is safe to human beings and has no effect on a rice plant; and the derivative of the miRNA obtained by modification also retains the effects and characteristics of the miRNA, can be used as a novel insecticide for safely and effectively preventing and controlling brown planthopper, can be directly used on a transgenic plant, and has a practical application value for brown planthopper prevention and control.

### TECHNICAL FIELD

The present disclosure belongs to the field of insect prevention and control technology, and particularly relates to a miRNA, a derivative and use thereof.

### SEQUENCE LISTING

The instant application contains a sequence listing that has been submitted in XML format via PATENT CENTER and is hereby incorporated by reference in its entirety. Said XML copy, created on Mar. 3, 2023, is entitled SEQUENCELISTING_XML.xml and is 23 KB in size.

### BACKGROUND

RNA interference (RNAi), a highly conserved cellular mechanism, is first found in nematodes (Caenorhabditis elegans). Since the first report, the RNAi has rapidly become a powerful reverse genetics tool for studying gene function, regulation and interaction on cell and tissue levels. Moreover, the RNAi has also exhibited great application value and potential in the field of pest prevention and control.

In insects and other species, there exist three different small RNAs (sRNAs) known to be able to induce three different types of RNAi pathways at present, which are respectively siRNA, miRNA and piRNA. Among them, the siRNA-mediated and miRNA-mediated post-transcriptional gene silencing (PTGS) is currently the main research means in the field of pest prevention and control. The siRNA has a length ranging from 19 to 21 bp, and is processed from long exogenous or endogenous dsRNA. The siRNA binds to a target mRNA in a highly specific binding mode, and further degrades the target mRNA. The miRNA is a type of single-stranded RNA with a length ranging from 19 to 24 bp, which is derived from endogenous primary miRNA (pri-miRNA) and forms small single-stranded RNA through processing, and has main functions of mediating the degradation of the target mRNA and inhibiting the expression of the target gene in animals and plants. Different from the siRNA, the miRNA binds to the target mRNA depending on a seed region sequence in animals, which means that binding results between the miRNA and the target mRNA are diverse. Generally speaking, based on action mechanisms of the siRNA and the miRNA, the effect of inhibiting important genes of a target pest may be exerted, and so as to achieve the purpose of pest prevention and control.

Brown planthopper is a monophagous Hemiptera insect with a habit of long-distance migration, and is currently a primary pest for a rice plant. Brown planthopper mainly sucks phloem juice of the rice plant by a piercing-sucking mouthpart, and may spread a variety of diseases on the rice plant. At present, many target genes for preventing and controlling the brown planthopper have been developed, but there are few miRNA useful to control the brown planthopper, and there is no report about preventing and controlling the brown planthopper with a form of plant-derived miRNA.

### SUMMARY

The present disclosure aims to solve at least one of the above-mentioned technical problems existing in the prior art. Therefore, the present disclosure provides a miRNA, which is effective in the insect prevention and control.

The present disclosure further provides a derivative of the miRNA above.

The present disclosure further provides a preparation method for the derivative of the miRNA above.

The present disclosure further provides a biological material associated with the miRNA above and the derivative of the miRNA above.

The present disclosure further provides use of the miRNA above, the derivative of the miRNA above and the biological material above.

The present disclosure further provides a method for insect prevention and control.

In a first aspect of the present disclosure, a miRNA is provided, comprising a nucleic acid sequence as shown in SEQ ID NO. 1 or a nucleic acid sequence obtained by modifying, substituting, deleting or adding at least one base to the nucleic acid sequence as shown in SEQ ID NO. 1.

In some embodiments of the present disclosure, the miRNA is a soybean (Glycine max)-derived miRNA.

In a second aspect of the present disclosure, a derivative of the miRNA is provided, comprising a nucleic acid sequence as shown in any one of SEQ ID NO. 2 and SEQ ID NO. 3 or a nucleic acid sequence obtained by modifying, substituting, deleting or adding at least one base to the nucleic acid sequence as shown in any one of SEQ ID NO. 2 and SEQ ID NO. 3.

In a third aspect of the present disclosure, a preparation method for the derivative of the miRNA is provided, wherein the method comprises: constructing the nucleic acid sequence of the miRNA into a vector to obtain a recombinant vector; introducing the recombinant vector into Escherichia coli (E. coli) to obtain a recombinant Escherichia coli; and inducing the expression of the recombinant Escherichia coli, and performing disruption to obtain the derivative of the miRNA.

In some embodiments of the present disclosure, the vector is a L4440 vector.

In some embodiments of the present disclosure, the Escherichia coli (E. coli) is at least one selected from the group consisting of HT115, DH5a and BL21.

In some embodiments of the present disclosure, the step of constructing the nucleic acid sequence of the miRNA into a vector comprises amplifying the sequence of the miRNA with primers.

In some embodiments of the present disclosure, the primers for amplifying the sequences of the miRNA have nucleotide sequences as shown in SEQ ID NO. 4 and SEQ ID NO. 5.

In some embodiments of the present disclosure, the disruption comprises ultrasonic cell disruption and lysozyme cell disruption.

In some embodiments of the present disclosure, after performing the disruption, the method further comprises extracting and purifying the miRNA. In some embodiments, the extracting and purifying comprises extracting a RNA by a Trizol method.

In a fourth aspect of the present disclosure, a biological material associated with the miRNA above and the derivative of the miRNA is provided, wherein the biological material is any one of 1) to 4):
1) a precursor of the miRNA above or the derivative of the miRNA;
2) a simulant of the miRNA above or the derivative of the miRNA;
3) a DNA molecule encoding the miRNA above, the derivative of the miRNA, or the precursor of the miRNA above or the derivative of the miRNA of 1); and
4) an expression cassette, a recombinant vector or a transgenic cell comprising the DNA molecule of 3).

In a fifth aspect of the present disclosure, use of the miRNA above and the derivative of the miRNA in insect prevention and control is provided.

In some embodiments of the present disclosure, the miRNA above and the derivative of the miRNA is for use in killing an insect body and/or inhibiting the growth of the insect body.

In some embodiments of the present disclosure, the insect is a rice pest.

In some embodiments of the present disclosure, the rice pest is brown planthopper.

According to a preferred embodiment of the present disclosure, the present disclosure has at least the following beneficial effects.

The miRNA is a Glycine max-derived Gma-miR482a (NR_048614.1), and may target to a plurality of different reported lethal target genes of the brown planthopper. By injecting the artificially synthesized Gma-miR482a, and the derivatives sRNA482a-228 and sRNA482a-542 of the Gma-miR482a into the insect body respectively, and the survival rate in 7 days is observed significantly decrease compared to that of the control group. Since the Gma-miR482a is a Glycine max-derived miRNA, and Glycine max is a daily food of human beings, this fragment is safe for human beings, may be used as a safe and effective novel insecticide for preventing and controlling the brown planthopper, and may be directly applied to a transgenic rice plant for pest prevention and control. Moreover, the Gma-miR482a is a specific miRNA of Leguminosae, so that the Gma-miR482a, and the sRNA482a-228 and the sRNA482a-542 are safe to the rice plant, and would not affect the growth and development of the rice plant.

In some embodiments of the present disclosure, the miRNA above and the derivative of the miRNA is for use in killing an insect body and/or inhibiting the growth of the insect body.

In some embodiments of the present disclosure, the insect is a rice pest.

In some embodiments of the present disclosure, the rice pest is brown planthopper.

The miRNA (Gma-miR482a) derived from the Glycine max and the derivatives sRNA482a-228 and sRNA482a-542 of the Gma-miR482a have an inhibiting effect on the growth of the rice pest—brown planthopper, which is a monophagous Hemiptera insect. Since the Gma-miR482a is derived from Glycine max which is a major crop, and the sRNA482a-228 and the sRNA482a-542 are derived from the Gma-miR482a, the sRNA482a, the sRNA482a-228 and the sRNA482a-542 are safe to human beings, may be used as novel safe and effective insecticide for preventing and controlling the brown planthopper, and may be directly applied to a transgenic plant, thus having a practical application value for preventing and controlling the brown planthopper.

In some embodiments of the present disclosure, the miRNA above and the derivative of the miRNA is for use in preparation of an insecticide.

In some embodiments, an insecticide is provided, comprising the miRNA above, the derivative of the miRNA or the biological material. Preferably, the insecticide further comprises a surfactant.

In some embodiments of the present disclosure, the surfactant is Tween 80.

In some embodiments of the present disclosure, the concentration of the Tween 80 ranges from 1 w/v % to 10 w/v %; preferably, the concentration of the Tween 80 ranges from 2.5 w/v % to 10 w/17%; and more preferably, the concentration of the Tween 80 ranges from 5 w/v % to 10 w/v %.

In a sixth aspect of the present disclosure, a method for insect prevention and control is provided, which comprises the following steps: introducing the miRNA above or the derivative of the miRNA into an insect or spraying the insecticide above onto a plant.

In some embodiments of the present disclosure, the step of introducing is implemented by injecting.

In some embodiments of the present disclosure, the step of introducing may be implemented by spraying.

In some embodiments of the present disclosure, the plant is a rice plant.

### BRIEF DESCRIPTION OF THE DRAWINGS

The present disclosure is further described hereinafter with reference to the drawings and the embodiments, wherein:

FIG. 1 shows an alignment between the sequence information of the Gma-miR482a in Embodiment 1 according to the present disclosure and a Vigna unguiculata transcriptome;

FIG. 2 shows an alignment between the sequence information of the Gma-miR482a in Embodiment 1 according to the present disclosure and a Glycine max transcriptome;

FIG. 3A shows the survival rates from 1^{st} day to 7^{th} day of the brown planthopper injected with the Gma-miR482a and PBS, and FIG. 3B is a morphologic image on 48 h of the brown planthopper injected with the Gma-miR482a;

FIG. 4 is a schematic diagram showing the construction of a vector L4440-Gma-miR482a in Embodiment 2 according to the present disclosure;

FIG. 5 is a profile of a plasmid L4440 in Embodiment 2 according to the present disclosure;

FIG. 6 shows the survival rates from 1^{st} day to 7^{th} day of the brown planthopper injected with a total RNA extracted by a Trizol method after a HT115 strain containing a L4440 empty vector being inducted (sRNACK), a total RNA extracted by the Trizol method after a HT115 strain containing a L4440-Gma-miR482a vector being inducted (sRNA482a-Trizol), a total RNA extracted by an ultrasonic+Trizol method after the HT115 strain containing the L4440-Gma-miR482a vector being inducted (sRNA482a-Ultrasonication) and a phosphate buffer solution (PBS) respectively in Embodiment 2 according to the present disclosure;

FIG. 7 shows a detection result of the inhibiting effect of the Gma-miR482a and sRNA482a-228 on α-N-acetylgalactosaminidase, JH-acid-O-methyltransferase, kayak, AP-1, krh1, CP16.5, FAD, Aminopeptidase Q, P450 4c3 and P450 4c1 genes in Embodiment 3 according to the present disclosure;

FIG. 8 is a graph showing the insecticidal effect on brown planthopper by injecting with the artificially synthesized Gma-miR482a, sRNA482a-228 and sRNA482a-542, and the phosphate buffer solution (PBS) in Embodiment 3 according to the present disclosure;

FIG. 9 is an image showing that Tween 80 in a Test Example according to the present disclosure reduces a liquid tension on a surface of the brown planthopper;

FIG. 10 is an image showing that the Tween 80 in a Test Example according to the present disclosure is beneficial for adhering a fluorescent label to the surface of the brown planthopper;

FIG. 11A shows a detection result of the insecticidal effect of the Tween 80 in a Test Example according to the present disclosure at a spraying amount of 3 mL, and FIG. 11B shows a detection result of the insecticidal effect of the Tween 80 in a Test Example according to the present disclosure at a spraying amount of 8 mL;

FIG. 12 shows a detection result of the insecticidal effect of sRNA482a (sRNA482a is generated by induction of the L4440-Gma-miR482a vector in the HT115 strain and extracted by a Trizol method)+2.5% Tween 80, a brown planthopper pesticide nitenpyram·pymetrozine and L4440 empty vector+2.5% Tween 80 in a Test Example according to the present disclosure;

FIG. 13 shows a detection result of the outdoor insecticidal effect of the sRNA482a+2.5% Tween 80 and the brown planthopper pesticide nitenpyram·pymetrozine on the brown planthopper in January in a Test Example according to the present disclosure;

FIG. 14 shows a detection result of the outdoor insecticidal effect of the sRNA482a+2.5% Tween 80 and the brown planthopper pesticide nitenpyram·pymetrozine on the brown planthopper in April in a Test Example according to the present disclosure;

FIG. 15 shows a detection result of the outdoor insecticidal effect of the sRNA482a+2.5% Tween 80 and the brown planthopper pesticide nitenpyram·pymetrozine on the brown planthopper in July in a Test Example according to the present disclosure;

FIG. 16 shows a detection result of the outdoor insecticidal effect of the sRNA482a+2.5% Tween 80 and the brown planthopper pesticide nitenpyram·pymetrozine on the brown planthopper in October in a Test Example according to the present disclosure;

FIG. 17 shows a detection result of the comprehensive outdoor insecticidal effect of the sRNA482a+2.5% Tween 80 and the brown planthopper pesticide nitenpyram·pymetrozine on the brown planthopper throughout a year in a Test Example according to the present disclosure;

FIG. 18 is a phenogram showing a tested rice plant in a Test Example according to the present disclosure;

FIG. 19 shows the effect of the sRNA482a+2.5% Tween 80 on the growth of a plant in a Test Example according to the present disclosure; and

FIG. 20 shows the effect of the sRNA482a+2.5% Tween 80 on the growth of a plant in a Test Example according to the present disclosure.

### DETAILED DESCRIPTION

The concept and the technical effect of the present disclosure will be clearly described in detail hereinafter with reference to exemplary implementations, for the convenience of understanding the purpose, the features and the effects of the present disclosure. Apparently, the described implementations are only part but not all of the embodiments of the present disclosure. Based on the implementations of the present disclosure, other implementations obtained by those skilled in the art without creative labor are all within the scope of protection of the present disclosure. Unless otherwise specified, all test methods used in the implementations are conventional methods; and unless otherwise specified, all materials and reagents used are commercially available.

In the description of the present disclosure, the descriptions of the reference terms “one embodiment”, “some embodiments”, “schematic embodiments”, “examples”, “specific examples”, or “some examples” refer to that the specific features, structures, materials, or characteristics described in the embodiment or example are included in at least one embodiment or example of the present disclosure. In the specification, the schematic description of the above terms does not necessarily mean the same embodiment or example. Moreover, the specific features, structures, materials or characteristics described may be combined into any one or more embodiments or examples in a suitable manner.

In order to screen an insect-resistant miRNA capable of preventing and controlling brown planthopper, but also being safe and environmentally friendly, a plant-derived miRNA library was used as a small-molecule library, and reported lethal genes of the brown planthopper were screened as a target gene library, then a target gene of the brown planthopper targeted by the plant-derived miRNA was predicted. It is found through the prediction that, there are 33 lethal target genes potentially targeted by the Gma-miR482a. The survival rate of nymph of brown planthopper injected with the artificially synthesized Gma-miR482a is significantly decreased in 7 days compared to a control group, which demonstrates that the Gma-miR482a has a potential insect-resistant value. Due to a high cost of artificial synthesis, in order to solve the problems of practical production and field application, the Gma-miR482a was constructed into a L4440 vector (a vector used for producing a long double-stranded RNA), and a small-molecule RNA (sRNA) was produced by a bacterial system capable of being produced industrially. Through the experimental verification, the sRNA482a basically retains the downstream gene and the insect-resistant effect of the Gma-miR482a. Moreover, through the prediction, the Gma-miR482a has no homology domain of consecutive 18 bp or above with a rice transcriptome, so that it can be considered that the Gma-miR482a would not affect the expression of a rice gene.

The Gma-miR482a is derived from Glycine max, which belongs to one kind of staple food plants for human beings, and is safe for both human beings and a rice plant. Therefore, the direct spraying of the Gma-miR482a, sRNA482a-228 and sRNA482a-542 on transgenic plants would have practical application values for pest prevention and control.

### Embodiment 1

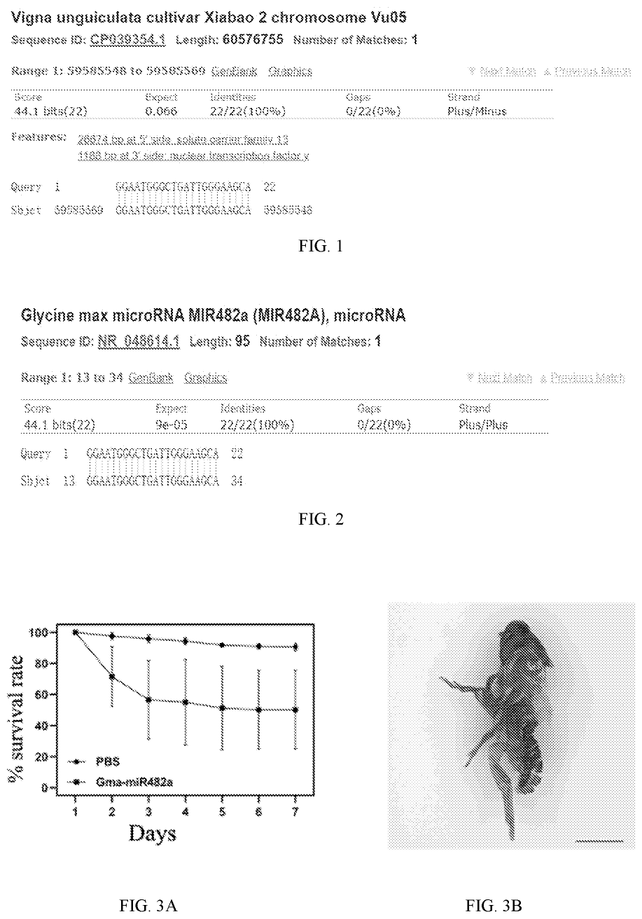

The embodiment provides a Gma-miR482a comprising a nucleic acid sequence of GGAATGGGCTGATTGGGAAGCA (SEQ ID NO. 1), and is derived from Glycine max. The alignments between the sequence of the Gma-miR482a with a Vigna unguiculata transcriptome and a Glycine max transcriptome are respectively shown in FIG. 1 and FIG. 2. The Gma-miR482a is used in preventing and controlling the brown planthopper.

1. Research on mechanism of Gma-miR482a in preventing and controlling brown planthopper

A lethal gene of the brown planthopper already reported was screened, a 3′UTR sequence of the lethal gene was extracted, and intersection analysis was carried out by using miRNA data obtained from the Glycine max, and thus the Gma-miR482a was found. Then, installation packages of software for Linux environment were respectively downloaded from websites of miRNA target gene prediction software, comprising PITA (https://genie.weizmann.ac.il/pubs/mir07/mir07 exe.html), miRanda (http://www.microrna.org/microrna/home.do) and RNAhybrid (https://bibiserv.cebitec.uni-bielefeld.de/rnahybrid/), and were further decompressed and installed. The sequence of the Gma-miR482a and the 3′UTR sequence of the lethal target gene of the brown planthopper were called in a Linux system to perform target gene prediction.

Prediction results are shown in Table 1 below:

TABLE 1
Prediction of binding site between Gma-miR482a and
3′UTR of reported lethal gene of brown planthopper

| | Initial | End | | |
|---|---|---|---|---|
| | Binding | Binding | | |
| | Site of | Site of | Stem- | |
| | 3′UTR | 3′UTR | loop | |
| Target Genes Name | (start) | (end) | structure | ddG |
| Ras-like family small | 272 | 264 | 0 | −8.4 |
| GTPases-Arf3 | | | | |
| Ras-like family small | 79 | 71 | 0 | −6.12 |
| GTPases-Arf3 | | | | |
| Ras-like family small | 62 | 54 | 0 | −7.73 |
| GTPases-Arf6 | | | | |
| Ras-like family small | 145 | 137 | 0 | 5.4 |
| GTPases-Arf6 | | | | |
| Argonaute-1 | 1718 | 1710 | 0 | −11.79 |
| Argonaute-1 | 1081 | 1074 | 0 | −7.32 |
| Argonaute-1 | 1788 | 1781 | 0 | −4.47 |
| Argonaute-1 | 685 | 678 | 0 | −4.33 |
| Argonaute-1 | 891 | 884 | 0 | 2.65 |
| Calcium/calmodulin- | 640 | 632 | 0 | −14.87 |
| dependent-protein-kinase- | | | | |
| type-II | | | | |
| Calcium/calmodulin- | 540 | 532 | 0 | −13.89 |
| dependent-protein-kinase- | | | | |
| type-II | | | | |
| Calcium/calmodulin- | 1353 | 1346 | 0 | −4.44 |
| dependent-protein-kinase- | | | | |
| type-II | | | | |
| Calcium/calmodulin- | 1251 | 1244 | 0 | −1.35 |
| dependent-protein-kinase- | | | | |
| type-II | | | | |
| Calcium/calmodulin- | 1302 | 1294 | 0 | −1.33 |
| dependent-protein-kinase- | | | | |
| type-II | | | | |
| Calmodulin | 1446 | 1440 | 0 | −9.13 |
| Calmodulin | 1238 | 1232 | 0 | −9 |
| C-terminal binding | 1587 | 1580 | 0 | −8.88 |
| protein2 | | | | |
| C-terminal binding | 1340 | 1333 | 0 | −12.22 |
| protein56 | | | | |
| C-terminal binding | 944 | 936 | 0 | −2.55 |
| protein56 | | | | |
| C-terminal binding | 956 | 948 | 0 | −11.95 |
| protein61 | | | | |
| C-terminal binding | 1297 | 1289 | 0 | −9.56 |
| protein61 | | | | |
| C-terminal binding | 582 | 574 | 0 | 0.6 |
| protein61 | | | | |
| C-terminal binding | 372 | 364 | 0 | 2.05 |
| protein61 | | | | |
| C-terminal binding | 827 | 819 | 0 | 4.16 |
| protein61 | | | | |
| C-terminal binding | 968 | 960 | 0 | −9.5 |
| protein62 | | | | |
| C-terminal binding | 1932 | 1924 | 0 | −5.29 |
| protein62 | | | | |
| C-terminal binding | 1615 | 1609 | 0 | −3.49 |
| protein62 | | | | |
| C-terminal binding | 1087 | 1079 | 0 | −4.58 |
| protein64 | | | | |
| C-terminal binding | 1134 | 1126 | 0 | −14.55 |
| protein69 | | | | |
| C-terminal binding | 1686 | 1678 | 0 | −5.16 |
| protein69 | | | | |
| C-terminal binding | 608 | 601 | 0 | 0.48 |
| protein69 | | | | |
| C-terminal binding | 1336 | 1329 | 0 | −11.4 |
| protein73 | | | | |
| C-terminal binding | 942 | 934 | 0 | −2.57 |
| protein73 | | | | |
| C-terminal binding | 1966 | 1958 | 0 | −8.08 |
| protein83 | | | | |
| C-terminal binding | 253 | 247 | 0 | −5.07 |
| protein83 | | | | |
| C-terminal binding | 1328 | 1320 | 0 | −1.93 |
| protein83 | | | | |
| C-terminal binding | 683 | 675 | 0 | −11.52 |
| proteine | | | | |
| C-terminal binding | 1319 | 1311 | 0 | −9.61 |
| proteine | | | | |
| C-terminal binding | 760 | 752 | 0 | −1.12 |
| proteinn-7 | | | | |
| C-terminal binding | 424 | 416 | 0 | −0.75 |
| proteinn-7 | | | | |
| ecdysone-induced-protein- | 972 | 965 | 0 | −11.73 |
| 93 | | | | |
| Egf-like-protein | 1651 | 1643 | 0 | −4.88 |
| Egf-like-protein | 1488 | 1480 | 0 | −4.62 |
| Egf-like-protein | 968 | 960 | 0 | 1.83 |
| Elongation-factor-1-alph | 161 | 155 | 0 | −17.56 |
| Elongation-factor-1-alph | 1224 | 1216 | 0 | −13.22 |
| Elongation-factor-1-alph | 107 | 99 | 0 | −7.55 |
| Endoribonuclease-Dcr-1 | 940 | 932 | 0 | −5.78 |
| Endoribonuclease-Dcr-1 | 1259 | 1251 | 0 | 0.72 |
| Fatty-acyl-coa | 992 | 984 | 0 | −13.2 |
| Fatty-acyl-coa | 1197 | 1189 | 0 | −11.41 |
| β-N-acetylhexosaminidase | 1311 | 1305 | 0 | −12.71 |
| 4 | | | | |
| β-N-acetylhexosaminidase | 1983 | 1977 | 0 | −5.69 |
| 4 | | | | |
| β-N-acetylhexosaminidase | 1353 | 1345 | 0 | −4.51 |
| 4 | | | | |
| Krüppel homolog 1 b | 867 | 859 | 0 | −10.84 |
| Krüppel homolog 1 b | 1691 | 1683 | 0 | −5.15 |
| Keratin 9 | 845 | 837 | 0 | −9.51 |
| Vanilloid-type transient | 82 | 76 | 0 | −12.04 |
| receptor potential channel | | | | |
| Nan | | | | |
| C-terminal binding | 823 | 815 | 0 | −10.6 |
| protein2 | | | | |
| NompCchannel | 968 | 962 | 0 | 2.48 |
| Phosphoglucomutase 1 | 469 | 462 | 0 | −3.64 |
| Phosphoglucomutase 1 | 1697 | 1689 | 0 | −1.34 |
| Phosphoglucomutase 21 | 1993 | 1986 | 0 | −7.61 |
| Phosphoglucomutase 21 | 51 | 43 | 0 | −5.81 |
| Phosphoglucomutase 21 | 1710 | 1702 | 0 | −5.04 |
| Ras-like family small | 1945 | 1937 | 0 | −18.19 |
| GTPases-Rab2 | | | | |
| Ras-like family small | 1145 | 1137 | 0 | −8.73 |
| GTPases-Rab2 | | | | |
| Ras-like family small | 56 | 48 | 0 | −8.46 |
| GTPases-Rab2 | | | | |
| Ras-like family small | 135 | 127 | 0 | −11.89 |
| GTPases-Rab7 | | | | |
| Ras-like family small | 111 | 103 | 0 | −6.25 |
| GTPases-Rab7 | | | | |
| Ras-like family small | 296 | 290 | 0 | −8.9 |
| GTPases-Rac | | | | |
| Catalytic subunit 3A of the | 1643 | 1637 | 0 | −6.67 |
| oligosaccharyltransferase | | | | |
| Catalytic subunit 3A of the | 1898 | 1890 | 0 | 1.37 |
| oligosaccharyltransferase | | | | |
| Tyrosine 3- | 1720 | 1712 | 0 | −4.72 |
| monooxygenase | | | | |
| Tyrosine 3- | 863 | 855 | 0 | −13.22 |
| monooxygenase | | | | |

Results in Table 1 shows that the Gma-miR482a may target to 3′UTR sites of a plurality of lethal target genes of the brown planthopper.

2. Verification of insecticidal effect of Gma-miR482a on brown planthopper

The 5^{th} instar nymphs of brown planthopper were divided into 2 groups, with 150 nymphs in each group. The 5^{th} instar nymphs of brown planthopper were injected with 0.5 μg of artificially synthesized Gma-miR482a in 50 nL PBS (experimental group) and 50 nL phosphate buffer solution (PBS) (control group) respectively. The nymphs after injection were fed on rice plants at an early tillering stage, and the plants were each separated by an air-permeable plastic cover. The survival rate was counted every 24 hours. The experiment was carried out in triplicate.

The observed results are shown in FIG. 3A and FIG. 3B, wherein FIG. 3A shows the survival rates of the rice brown planthopper injected with the Gma-miR482a and PBS, and FIG. 3B shows a morphologic image of the brown planthopper injected with the Gma-miR482a. It can be seen from the figures that, 7 days after injection with the Gma-miR482a, the survival rate of the experimental group is 51.2%, while the survival rate of the control group is 92%. The results show that the Gma-miR482a can significantly decrease the survival rate of the brown planthopper, so that the brown planthopper might die due to failed molting.

### Embodiment 2

The embodiment provides a derivative sRNA482a of a Gma-miR482a.

1. Preparation method for derivative sRNA482a of Gma-miR482a

The preparation method for the derivative sRNA482a of the Gma-miR482a comprised the following steps.

(1) An L4440 empty vector (purchased from Beijing TIANDZ Gene Technology Co., Ltd.) was double-digested with Kpn I and Bgl II, all digestion sites between two T7 promoters were removed, then a pair of primers T7-482a-F and T7-482a-R was used as a template to amplify a fragment containing partial sequences of the T7 promoters at two ends and a sequence of the Gma-miR482a, and the amplified fragment containing the sequence of the Gma-miR482a was assembled between the two T7 promoters by homologous recombination to construct a L4440-Gma-miR482a vector. The primers of the homologous recombination comprised:

T7-482a-F:
(SEQ ID NO. 4)
TAATACGACTCACTATAGGGGAATGGGCTGATTGGGAAGCA;
and
T7-482a-R:
(SEQ ID NO. 5)
TAATACGACTCACTATAGGTGCTTCCCAATCAGCCCATTCC.

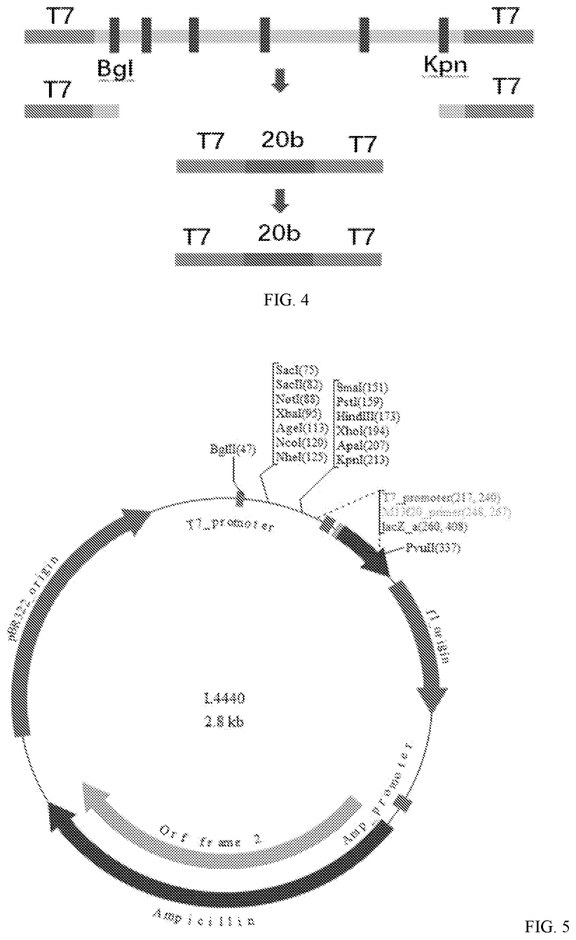

The construction procedure for the L4440-Gma-miR482a vector is shown in FIG. 4, and a plasmid profile of the L4440 is shown in FIG. 5.

(2) After the constructed L4440-Gma-miR482a vector was introduced into a HT115 strain, the recombinant HT115 strain was induced to express, which specifically comprised the following steps: activating the HT115 containing the L4440-Gma-miR482a at 37° C. overnight, adding the activated HT115 into a culture medium comprising ampicillin (100 ng/ml) and tetracycline (12.5 ng/ml) at a volume ratio of 1:100, expanding the culture at 37° C. until the OD value of the culture solution was then adding IPTG (0.1 mol/L) at a volume ratio of 1:1,000 to induce the synthesis of sRNA for 6 hours, and then collecting bacteria by 11,000 rpm of centrifuge. Based on a concentration obtained by suspending 700 ml of the culture solution into 16 ml, the recombinant HT115 strain after induced expression was obtained.

The recombinant HT115 strain after induced expression was disrupted under an output power of ultrasonic wave ranging from 20 W to 22 W for 28 minutes (stopping for 10 seconds after working for 10 seconds). The disrupted solution was extracted by a Trizol kit (purchased from Shanghai Sangon Biotech Co., Ltd.) to obtain the derivative sRNA482a of the Gma-miR482a synthesized with the bacteria (sRNA482a-Ultrasonication).

The recombinant HT115 strain after induced expression was disrupted by lysozyme, and the disrupted solution was extracted by a Trizol kit (purchased from Shanghai Sangon Biotech Co., Ltd.) to obtain a total RNA extracted by a Trizol method (sRNA482a-Trizol). Under the similar conditions, a total RNA was obtained by extracting with Trizol after a HT115 strain containing a L4440 empty vector being inducted and lysozyme-disrupted (sRNACK).

2. Verification of insecticidal effect of sRNA482a

The Effects of the sRNA482a generated by a bacterial system, which resulted from ultrasonically disrupting the bacteria and RNA extracted by a Trizol method, in preventing and controlling the brown planthopper were tested. The verification comprised the following steps: the 5^{th} instar nymphs of brown planthopper were injected with a total RNA extracted by a Trizol method after induction of the HT115 strain containing the L4440 empty vector (sRNACK), a total RNA extracted by the Trizol method after induction of the HT115 strain containing the L4440-Gma-miR482a vector (sRNA482a-Trizol), a total RNA extracted by an ultrasonic+Trizol method after induction of the HT115 strain containing the L4440-Gma-miR482a vector (sRNA482a-Ultrasonication) and a phosphate buffer solution (PBS) (control group) respectively, with an injection volume of 100 nL and an injection dose of 500 ng per nymph. The nymphs after injection were fed on rice plants at an early tillering stage, and the plants were each separated by an air-permeable plastic cover, and the survival rate was counted every 24 hours.

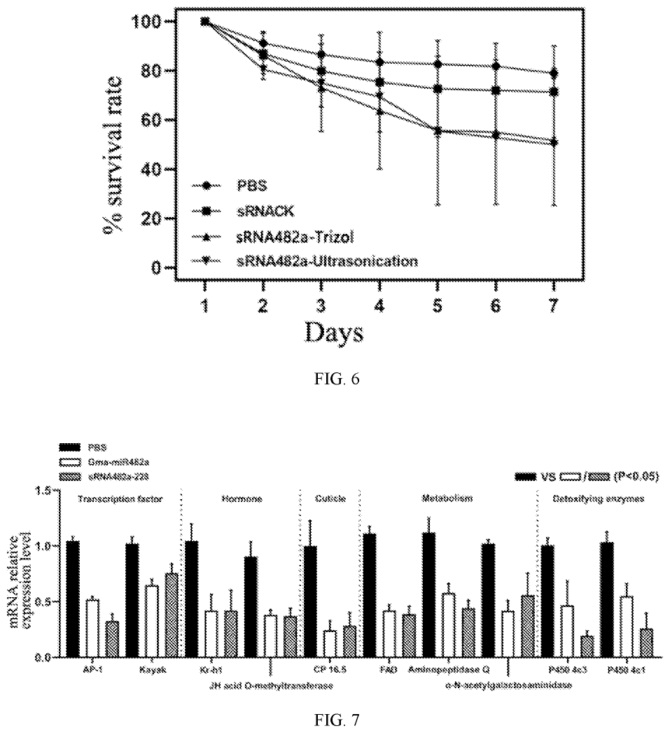

The experimental results are shown in FIG. 6, and it can be seen that, after 7 days, the insecticidal effect of the sRNA482a obtained by the ultrasonic+Trizol method and the Trizol method are similar, and the death rates of the brown planthopper are both higher than that of the control group.

### Embodiment 3

The embodiment provides derivatives sRNA482a-228 and sRNA482a-542 of Gma-miR482a, wherein the nucleotide sequence of the sRNA482a-228 is ACTCACTATAGGGGAATGGGCTGATTGGGAAGCACCTATAGTGAGT (SEQ ID NO. 2); and a nucleotide sequence of the sRNA482a-542 is GGCTGATTGGGAAGCACCTATAGTGAGTCG (SEQ ID NO. 3).

1. Preparation of the derivatives sRNA482a-228 and sRNA482a-542 of the Gma-miR482a comprised the following steps: performing sequencing analysis on the derivative sRNA482a (sRNA482a-Trizol) generated by the bacterial system obtained in Embodiment 2, and the sequencing reads of two sRNAs (the sRNA482a-228 and the sRNA482a-542) are shown in Table 2. The sequence of the sRNA482a-228 contains a full sequence of the Gma-miR482a and partial sequences of T7 promoters at two ends of a vector, and the sequence of the sRNA482a-542 contains a partial sequence of the Gma-miR482a and partial sequence of the T7 promoters.

TABLE 2

| SRNA | Sequence | Count |
|---|---|---|
| SRNA482a- | ACTCACTATAGGGGAATGGGCTGATTGGGA | 1168 |
| 228 | AGCACCTATAGTGAGT | |
| | (SEQ ID NO. 2) | |
| SRNA482a- | GGCTGATTGGGAAGCACCTATAGTGAGTCG | 439 |
| 542 | (SEQ ID NO. 3) | |

2. Research on molecular mechanisms of Gma-miR482a and sRNA482a-228 in insecticidal action

Since the count value of the sRNA482a-542 only accounts for 37.59% of the count value of the sRNA482a-228 and the sRNA482a-542, the SRNA482a-228 was chosen as a main representative of the sRNA482a to study the insecticidal action mechanism. Through the transcriptome sequencing analysis on Gma-miR482a and sRNA482a-228 with an equivalent volume of PBS as control, it was found that Gma-miR482a up-regulated 291 genes and down-regulated 265 genes; and sRNA482a-228 up-regulated 455 genes and down-regulated 505 genes. Since negatively regulating dominates in regulation of genes by a small-molecule RNA, the down-regulated genes were analyzed, and it was found that almost all down-regulated genes by the Gma-miR482a appeared in the down-regulated genes by the sRNA482a-228, while most of the down-regulated genes only by the sRNA482a-228 were relatively non-lethal genes. It was indicated that the sRNA482a-228 mainly executed the insecticidal mechanism of the Gma-miR482a. The Genes intersecting in two transcriptomes mainly comprised: transcription factors kayak and AP-1, epidermal protein family gene, cytochrome P450 family gene, endocuticle structural glycoprotein gene, chitinase gene, juvenile hormone synthesis and juvenile hormone acid O-methyltransferase genes, Kruppel homologl (krh1) and Krueppel-like factor 10 genes. These were all important genes for the structure, growth and development, and management to a plant secondary substance of insect.

5^{th} instar nymphs of brown planthopper were injected with 0.5 μg of the artificially synthesized Gma-miR482a and sRNA482a-228, and PBS buffer solution (control group) respectively, with an injection volume of 100 mL. The inhibiting effects of the Gma-miR482a and the sRNA482a-228 on these above genes were tested by qRT-PCR, which was carried out using Taq Pro Universal SYBR QPCR Master Mix kit and primers shown in Table 3. The results of qRT-PCR are shown in FIG. 7, and the inhibiting effects of the Gma-miR482a and the sRNA482a-228 on α-N-acetylgalactosaminidase, JH-acid-O-methyltransferase, kayak, AP-1, krh1, CP16.5, FAD, Aminopeptidase Q, P450 4c3 and P450 4c1 genes can be seen from the figure.

TABLE 3

| Genes | Primers (5′-3′) |
|---|---|
| α-N-acetyl- | AAGTCAATCCATCGGGAGTCGTTA |
| galactosa- | (SEQ ID NO. 6) |
| minidase-qF | |
| α-N-acetyl- | ATATTTGCCTGAATGGGTTGTAAGG |
| galactosa- | (SEQ ID NO. 7) |
| minidase-qR | |
| Aminopep- | TATACGAAGATTGGCGGAGATGTTG |
| tidase Q-qF | (SEQ ID NO. 8) |
| Aminopep- | ATCTGGCCACCTTGATTATGAAAGA |
| tidase Q-qR | (SEQ ID NO. 9) |
| cp16.5-qF | CCTTGTTCTGAGCGCCTTGGT |
| | (SEQ ID NO. 10) |
| cp16.5-qR | CCCACGCGGTCGAACTTGA |
| | (SEQ ID NO. 11) |
| P450-4C1-qF | GCTACTGCTACCTGCCCTTCAGTT |
| | (SEQ ID NO. 12) |
| P450-4C1-qR | ACGTGGGTGGTCAGCTTGTAGTTC |
| | (SEQ ID NO. 13) |
| P450-4c3-qF | GGTCATCAAAGAAGTTCTGCGGTTA |
| | (SEQ ID NO. 14) |
| P450-4c3-qR | TCCTGCTGGGAACACTGTCGA |
| | (SEQ ID NO. 15) |
| FAD-qF | TACCCGCCACTGACGGAGTCT |
| | (SEQ ID NO. 16) |
| FAD-qR | GCGCCCACAATAACAAAGTCGTA |
| | (SEQ ID NO. 17) |
| JH-acid-O- | GGCAAGACGGCGAGACCAT |
| methyltrans- | (SEQ ID NO. 18) |
| ferase-qF | |
| JH-acid-O- | CATGTTCCACCATATTCGACGAAA |
| methyltrans- | (SEQ ID NO. 19) |
| ferase-qR | |
| krh1-qF | ACACGCCAGATAGAATAAGGGTCAA |
| | (SEQ ID NO. 20) |
| krh1-qR | GCAGCTTCACTCCTCTCATCTTTCC |
| | (SEQ ID NO. 21) |
| AP-1-qF | TTCTACGAGGAGGGTTCATTCAATC |
| | (SEQ ID NO. 22) |
| AP-1-qR | CACGTTTCGCGTCGTTGTGA |
| | (SEQ ID NO. 23) |
| kayak-qF | ACCTACCAGCCTGCCAGTTGTG |
| | (SEQ ID NO. 24) |
| kayak-qR | TCCATCAGCGAGTCGAAGTTGA |
| | (SEQ ID NO. 25) |

3. Verification of insecticidal effects of sRNA482a-228 and sRNA482a-542

Since the sRNA482a-228 and the sRNA482a-542 were contained in the small-molecule RNA obtained by the bacterial system, the sRNA482a-228 and the sRNA482a-542 were artificially synthesized respectively for verification of the insect-resistant effect of the both.

Experimental procedure: the 5^{th} instar nymphs of brown planthopper were divided into 4 groups, each with 150 nymphs, and each group were injected with 500 ng of artificially synthesized Gma-miR482a, sRNA482a-228, sRNA482a-542 and phosphate buffer solution (PBS) (control group) respectively, with an injection volume of 100 nL. The nymphs after injection were fed on rice plants at an early tillering stage, and the plants were each separated by an air-permeable plastic cover, and the survival rates were counted every 24 hours.

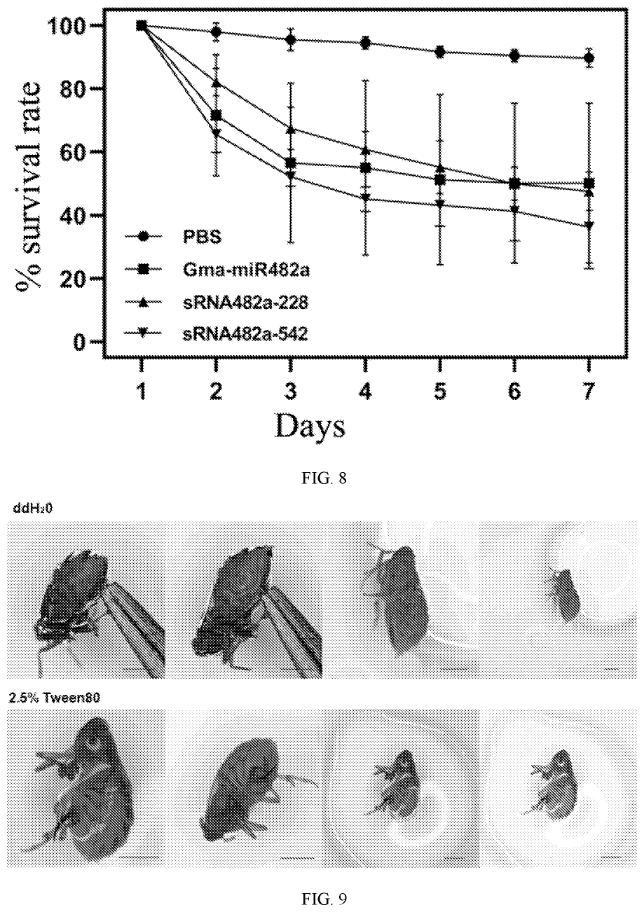

The experimental results are shown in FIG. 8, it can be seen that, after 7 days, the sRNA482a-542 have a better effect in preventing and controlling the rice brown planthopper compared to the Gma-miR482a, the Gma-miR482a and the sRNA482a-228 have the similar insecticidal effects, and all of them can significantly decrease the survival rate of the brown planthopper, so that the brown planthopper might die due to failed molting.

### Embodiment 4

The embodiment provides an insecticide, comprising 2.5% Tween 80 and the sRNA482a. The Tween 80 was added to an aqueous solution of sRNA482a (sRNA482a-Trizol) prepared in Embodiment 2, with a final concentration of 2.5%.

### Test Example

1. Effect Verification of Tween 80 on inhibition of survival of brown planthopper

(1) Reduction of liquid tension on surface of brown planthopper by Tween 80 so as to increase small-molecule RNA adhered to surface of brown planthopper

In order to promote the sRNA aqueous solution to adhere to the waxy surface of the insect, the surfactant Tween 80 was tested. The 5^{th} instar nymphs of brown planthopper were divided into 2 groups, each with 150 nymphs. A container containing 2.5% Tween 80, 15 μg/mL artificially synthesized Gma-miR482a and ddH₂O was used in the experimental group, and a container containing an equal amount of Gma-miR482a and ddH₂O was used in the control group. The 5^{th} instar nymphs of brown planthopper were put into the containers, and the experiment was repeated for 5 times in each group. The surface observation results after 6 hours are shown in FIG. 9, and it can be seen that the Tween 80 in the solution of the present disclosure can effectively reduce the liquid tension on the surface of the brown planthopper.

(2) Verification of improvement to aggregation effect of Gma-miR482a on insect surface by Tween 80

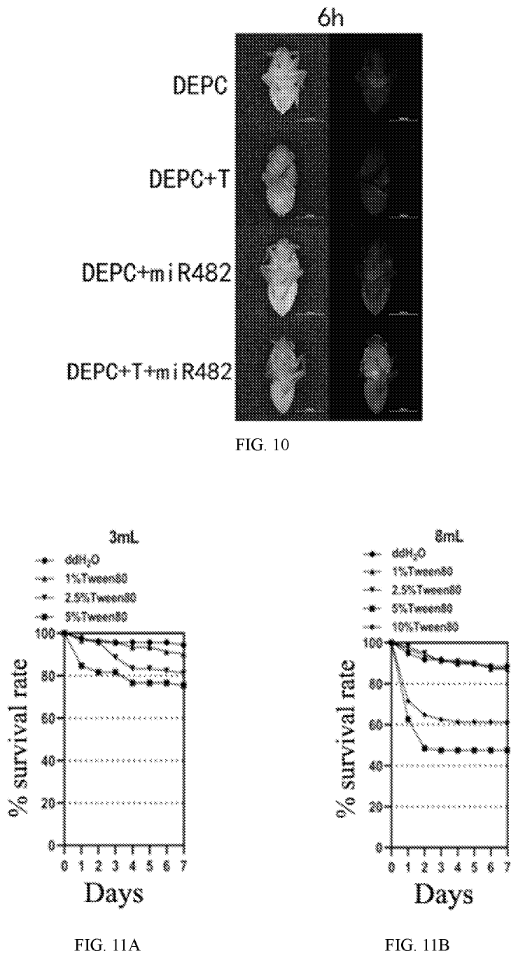

In order to prove whether the Tween 80 improves the aggregation of the Gma-miR482a on the surface of the brown planthopper after reducing the liquid tension on the insect surface, the artificially synthesized Gma-miR482a was labeled with green fluorescence (5′FAM). The 5^{th} instar nymphs of brown planthopper were divided into 4 groups, each with 150 nymphs. A container containing 2.5% Tween 80, 15 μg/mL Gma-miR482a and DEPC water was used in the experimental group, and the control group 1 (DEPC group) differed from the experimental group only in that the container did not contain 2.5% Tween 80 and the Gma-miR482a; the control group 2 (DEPC+T group) differed from the experimental group only in that the container did not contain the Gma-miR482a; and the control group 3 (DEPC+Gma-miR482a group) differed from the experimental group only in that the container did not contain 2.5% Tween 80. The 5^{th} instar nymphs of brown planthopper were put into the containers, and the experiment was repeated for 5 times in each group. Through scanning, the fluorescence detection results after 6 hours are shown in FIG. 10, and it can be seen that the Tween is beneficial for adhering the fluorescently labeled Gma-miR482a to the surface of the brown planthopper.

(3) Verification of insecticidal effect of Tween 80 with different concentrations on brown planthopper

The insecticidal effect of the Tween 80 was tested with the experiment comprising the following steps: dividing the 3^{rd} instar nymphs of brown planthopper into 9 groups, each with 150 nymphs, and spraying 3 mL of ddH₂O, 3 mL of 1% Tween 80, 3 mL of 2.5% Tween 80, 3 mL of 5% Tween 80, 8 mL of ddH₂O, 8 mL of 1% Tween 80, 8 mL of 2.5% Tween 80, 8 mL of 5% Tween 80 and 8 mL of 10% Tween 80 on the surfaces of the nymphs of brown planthopper respectively. The results after 6 hours are shown in FIG. 11, and it can be seen that the Tween 80 can effectively reduce the survival rate of the brown planthopper, and the insect-resistant effect of the Tween 80 is related to the concentration, which may be related to a damage caused by the Tween 80 to epidermis of the pest. Therefore, in order to avoid an influence on the insect-resistant effect of the small-molecule RNA and reduce a possible influence on environment, 2.5% Tween 80 with an effect close to that of ddH₂O in the control group was selected to prepare a sRNA preparation.

(4) Outdoor verification of insecticidal effect of sRNA482a on brown planthopper

The insecticide (sRNA482a+2.5% Tween 80) prepared in Embodiment 4 according to the present disclosure was used in a pilot test for pest prevention and control on 72 rice seedlings at an early tillering stage in an outdoor cement pond (4×1 m), wherein the sRNA482a (sRNA482a-Trizol) was prepared in Embodiment 2.

The 3^{rd} to 5^{th} instar nymphs of brown planthopper were divided into 3 groups, each with about 500 nymphs. The sRNA482a (sRNA482a-Trizol) was obtained by introducing the expression of a L4440-Gma-miR482a vector in a bacterial system, then performing cell disruption with lysozyme, and then extracting RNA with a Trizol kit. sRNA482a+2.5% Tween 80 was used in the experimental group with the dosage of the sRNA482a of about 30 mg for one cement pond; a brown planthopper pesticide nitenpyram·pymetrozine was used in the positive control group (with a concentration of g/L according to a recommended dosage); and RNA of the bacterial system (a total RNA obtained by extracting with Trizol after a bacterium containing a L4440 empty vector being inducted and disrupted (sRNACK))+2.5% Tween 80 was used in the negative control group, each group with the same spraying volume of 20 mL. The pilot test for pest prevention and control was carried out in the outdoor cement pond. The nymphs of the brown planthopper were put on rice plants at an early tillering stage, and the ponds were each separated by an air-permeable plastic cover. The preparations in the experimental group and the control groups were respectively sprayed on the nymphs of brown planthopper, and the survival rate was counted after 0 hour, 24 hours, 72 hours, 120 hours and 168 hours.

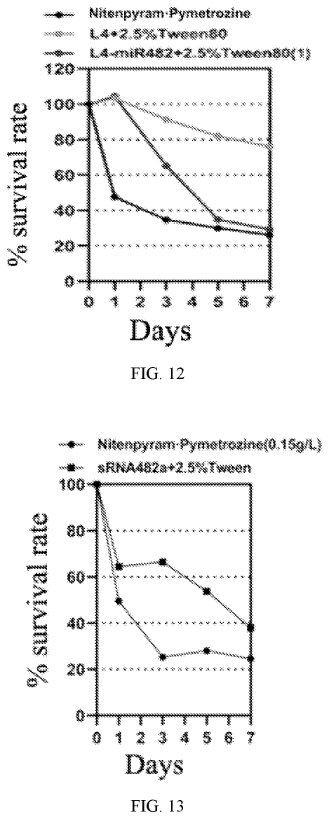

The experimental results are shown in FIG. 12, as can be seen, the insecticidal efficiency within 7 days after spraying the sRNA482a+2.5% Tween 80 is significantly higher than that of the negative control, and only about 2% to 10% lower than that of the brown planthopper pesticide nitenpy ram·pymetrozine.

(5) Comparison and verification of insecticidal effect of sRNA482a preparation and pesticide on brown planthopper

The insecticide prepared in Embodiment 4 according to the present disclosure was used in a pilot test for pest prevention and control in an outdoor cement pond, and compared with the pesticide, an outdoor insecticidal effect of the sRNA482a was evaluated.

The 3^{rd} instar nymphs of brown planthopper were divided into 2 groups, each with about 500 nymphs. The sRNA482a (sRNA482a-Trizol generated by the L4440-Gma-miR482a in the bacterium)+2.5% Tween 80 was used in the experimental group, and the brown planthopper pesticide nitenpyram·pymetrozine was used in the positive control group (with the concentration of 0.15 g/L according to the recommended dosage). In the pilot test for pest prevention and control in the outdoor cement pond, both the dosages of the insecticide of experimental group and the positive control were the same as those in (4) Outdoor verification of insecticidal effect of sRNA482a on brown planthopper. The nymphs of the brown planthopper were fed on rice plants at an early tittering stage, and the ponds were each separated by an air-permeable plastic cover. The experimental group and the control group were respectively sprayed on the nymphs of brown planthopper, and the survival rate was counted every 24 hours. The experiment was respectively carried out in January, April, July and October of the same year, so that the time points of the four experiments covered the whole year.

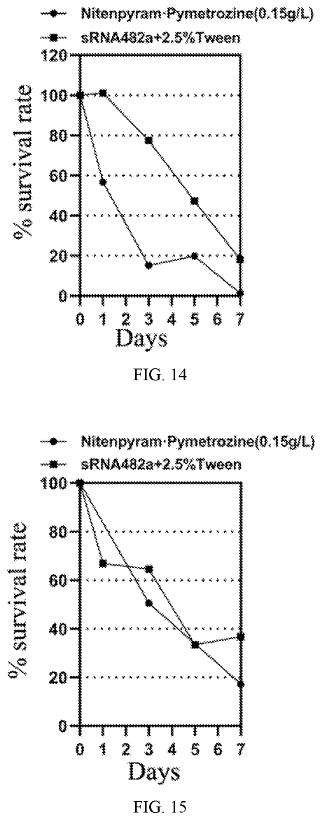

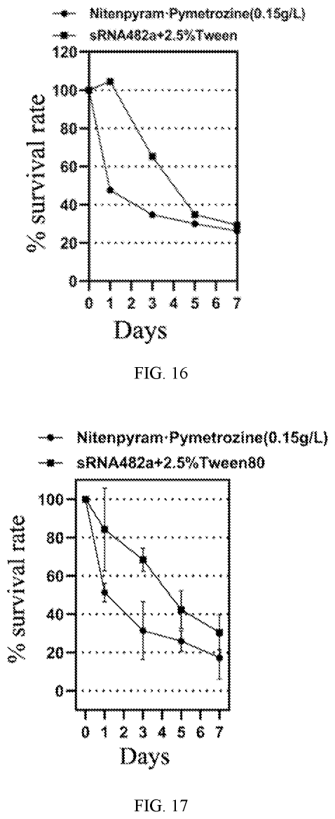

The experimental results are shown in FIG. 13 to FIG. 16, and it can be seen that the spraying of the sRNA482a+2.5% Tween in different seasons can effectively reduce the survival rate of the brown planthopper, and the sRNA482a+2.5% Tween achieve the similar insect-resistant effect with that of the brown planthopper pesticide nitenpyram·pymetrozine. FIG. 17 shows the average values of these four experiments, and the results are similar.

(6) Verification of phenotype of rice plants after spraying sRNA482a preparation on pests

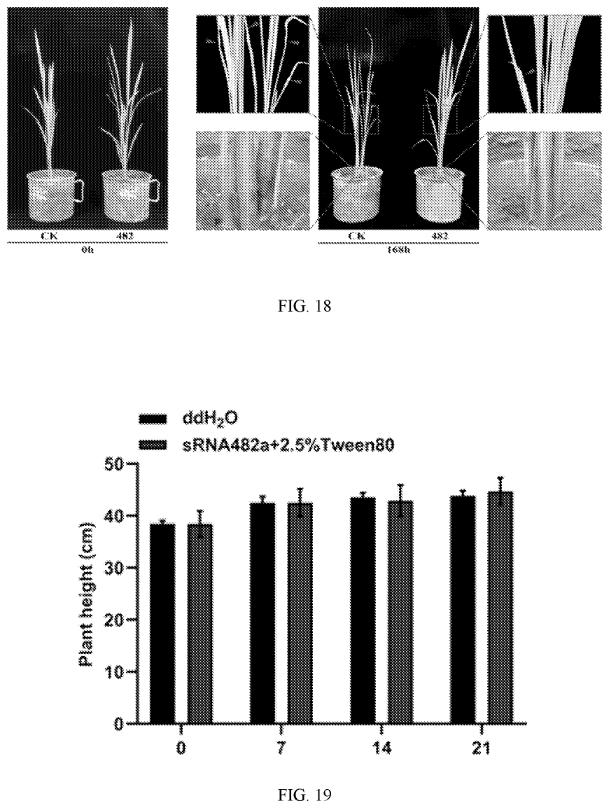

The rice plants were divided into 2 groups, the control group was sprayed with clear water, and the experimental group was sprayed with the sRNA482a+2.5% Tween 80 (the insecticide prepared in Embodiment 4), and 150 3^{rd} to 5^{th} instar nymphs of brown planthopper were inoculated on each rice plant. The insecticide was sprayed on the pests with a dosage same as that in the experiment in (4) Outdoor verification of insecticidal effect of sRNA482a on brown planthopper. As shown in FIG. 18, it can be seen that, after 7 days, leaves of rice seedlings in the control group already turn yellow, while leaves of rice seedlings sprayed with the sRNA482a are normal except one slightly yellow leaf (indicated by an arrow), which indicates that the invasion of the pests is reduced.

2. Verification of Safety Test
(1) Verification of phenotype of rice plants

The rice plants were divided into 2 groups, the control group was sprayed with clear water, and the experimental group was sprayed with the insecticide (sRNA482a+2.5% Tween 80) prepared in Embodiment 4 with a dosage about 2.5 times higher than the dosage of the insecticide sprayed on the pests in the experiment in (4) Outdoor verification of insecticidal effect of Embodiment 4. The insecticide was applied to roots of the rice plants once a week for 4 consecutive times.

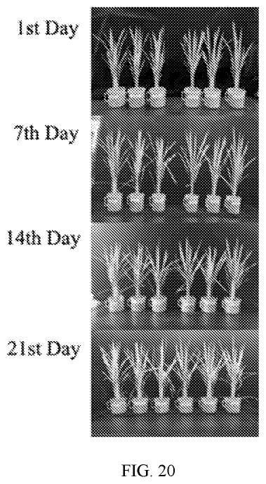

The phenotype and weight detection results of the rice plants three weeks after the treatments are shown in FIG. 19 to FIG. 20, and the results show that the sRNA482a+2.5% Tween 80 have no effect on the growth of the rice plants.

The embodiments of the present disclosure have been described in detail with reference to the drawings above, but the present disclosure is not intended to be limited by the above embodiments, and various changes may be made within the knowledge scope possessed by those skilled in the art without departing from the purpose of the present disclosure. In addition, the embodiments of the present disclosure and the features in the embodiments may be combined with each other without conflict.

1. A miRNA, comprising a nucleic acid sequence as shown in SEQ ID NO. 1 or a nucleic acid sequence obtained by modifying, substituting, deleting or adding at least one base to the nucleic acid sequence as shown in SEQ ID NO. 1.

2. A derivative of the miRNA according to claim 1, comprising a nucleic acid sequence as shown in any one of SEQ ID NO. 2 and SEQ ID NO. 3 or a nucleic acid sequence obtained by modifying, substituting, deleting or adding at least one base to the nucleic acid sequence as shown in any one of SEQ ID NO. 2 and SEQ ID NO. 3.

3. A preparation method for the derivative of the miRNA according to claim 2, comprising:
constructing a miRNA into a vector to obtain a recombinant vector; wherein the miRNA comprises a nucleic acid sequence as shown in SEQ ID NO. 1 or a nucleic acid sequence obtained by modifying, substituting, deleting or adding at least one base to the nucleic acid sequence as shown in SEQ ID NO. 1;
introducing the recombinant vector into Escherichia coli to obtain a recombinant Escherichia coli; and
inducing the expression of the recombinant Escherichia coli, and performing disruption to obtain the derivative of the miRNA.

4. A biological material associated with the miRNA according to claim 1, wherein the biological material is any one of 1) to 4):
1) a precursor of the miRNA;
2) a simulant of the miRNA;
3) a DNA molecule encoding the miRNA, or the precursor of the miRNA of 1); and
4) an expression cassette, a recombinant vector or a transgenic cell containing the DNA molecule of 3).

5. A biological material associated with the derivative of the miRNA according to claim 2, wherein the biological material is any one of 1) to 4):
1) a precursor of the derivative of the miRNA;
2) a simulant of the derivative of the miRNA;
3) a DNA molecule encoding the derivative of the miRNA, or the precursor of the derivative of the miRNA of 1); and
4) an expression cassette, a recombinant vector or a transgenic cell containing the DNA molecule of 3).

6. An insecticide, comprising the miRNA according to claim 1.

7. The insecticide according to claim 6, further comprising a surfactant.

8. The insecticide according to claim 7, wherein the surfactant is Tween 80, and the concentration of the Tween 80 ranges from 1 w/v % to 10 w/v %.

9. The insecticide according to claim 8, wherein the concentration of the Tween 80 ranges from 2.5 w/v % to 10 w/v %.

10. An insecticide, comprising the derivative of the miRNA according to claim 2.

11. The insecticide according to claim 10, further comprising a surfactant.

12. The insecticide according to claim 11, wherein the surfactant is Tween 80, and the concentration of the Tween 80 ranges from 1 w/v % to 10 w/v %.

13. A method for insect prevention and control, comprising: introducing the miRNA according to claim 1 into an insect.

14. The method according to claim 13, wherein the insect comprises a rice pest.

15. The method according to claim 14, wherein the rice pest comprises brown planthopper.

16. A method for insect prevention and control, comprising: introducing the derivative of the miRNA according to claim 2 into an insect.

17. The method according to claim 16, wherein the insect comprises a rice pest.

18. A method for insect prevention and control, comprising: spraying the insecticide according to claim 6 onto a plant.

19. The method according to claim 18, wherein the plant is a rice plant.

20. A method for insect prevention and control, comprising: spraying the insecticide according to claim 10 onto a plant.


SEQUENCE LISTING (25 sequences)

SEQ ID NO: 1 - DNA, 22 bp, synthetic construct
  source 1..22: mol_type = other DNA
ggaatgggctgattgggaagca

SEQ ID NO: 2 - DNA, 46 bp, synthetic construct
  source 1..46: mol_type = other DNA
actcactataggggaatgggctgattgggaagcacctatagtgagt

SEQ ID NO: 3 - DNA, 30 bp, synthetic construct
  source 1..30: mol_type = other DNA
ggctgattgggaagcacctatagtgagtcg

SEQ ID NO: 4 - DNA, 41 bp, synthetic construct
  source 1..41: mol_type = other DNA
taatacgactcactataggggaatgggctgattgggaagca

SEQ ID NO: 5 - DNA, 41 bp, synthetic construct
  source 1..41: mol_type = other DNA
taatacgactcactataggtgcttcccaatcagcccattcc

SEQ ID NO: 6 - DNA, 24 bp, synthetic construct
  source 1..24: mol_type = other DNA
aagtcaatccatcgggagtcgtta

SEQ ID NO: 7 - DNA, 25 bp, synthetic construct
  source 1..25: mol_type = other DNA
atatttgcctgaatgggttgtaagg

SEQ ID NO: 8 - DNA, 25 bp, synthetic construct
  source 1..25: mol_type = other DNA
tatacgaagattggcggagatgttg

SEQ ID NO: 9 - DNA, 25 bp, synthetic construct
  source 1..25: mol_type = other DNA
atctggccaccttgattatgaaaga

SEQ ID NO: 10 - DNA, 21 bp, synthetic construct
  source 1..21: mol_type = other DNA
ccttgttctgagcgccttggt

SEQ ID NO: 11 - DNA, 19 bp, synthetic construct
  source 1..19: mol_type = other DNA
cccacgcggtcgaacttga

SEQ ID NO: 12 - DNA, 24 bp, synthetic construct
  source 1..24: mol_type = other DNA
gctactgctacctgcccttcagtt

SEQ ID NO: 13 - DNA, 24 bp, synthetic construct
  source 1..24: mol_type = other DNA
acgtgggtggtcagcttgtagttc

SEQ ID NO: 14 - DNA, 25 bp, synthetic construct
  source 1..25: mol_type = other DNA
ggtcatcaaagaagttctgcggtta

SEQ ID NO: 15 - DNA, 21 bp, synthetic construct
  source 1..21: mol_type = other DNA
tcctgctgggaacactgtcga

SEQ ID NO: 16 - DNA, 21 bp, synthetic construct
  source 1..21: mol_type = other DNA
tacccgccactgacggagtct

SEQ ID NO: 17 - DNA, 23 bp, synthetic construct
  source 1..23: mol_type = other DNA
gcgcccacaataacaaagtcgta

SEQ ID NO: 18 - DNA, 19 bp, synthetic construct
  source 1..19: mol_type = other DNA
ggcaagacggcgagaccat

SEQ ID NO: 19 - DNA, 24 bp, synthetic construct
  source 1..24: mol_type = other DNA
catgttccaccatattcgacgaaa

SEQ ID NO: 20 - DNA, 25 bp, synthetic construct
  source 1..25: mol_type = other DNA
acacgccagatagaataagggtcaa

SEQ ID NO: 21 - DNA, 25 bp, synthetic construct
  source 1..25: mol_type = other DNA
gcagcttcactcctctcatctttcc

SEQ ID NO: 22 - DNA, 25 bp, synthetic construct
  source 1..25: mol_type = other DNA
ttctacgaggagggttcattcaatc

SEQ ID NO: 23 - DNA, 20 bp, synthetic construct
  source 1..20: mol_type = other DNA
cacgtttcgcgtcgttgtga

SEQ ID NO: 24 - DNA, 22 bp, synthetic construct
  source 1..22: mol_type = other DNA
acctaccagcctgccagttgtg

SEQ ID NO: 25 - DNA, 22 bp, synthetic construct
  source 1..22: mol_type = other DNA
tccatcagcgagtcgaagttga